(19) United States
(12) Patent Application Publication
(10) Pub. No.: US 2012/0055045 A1
(43) Pub. Date: Mar. 8, 2012

(54) ORTHOTIC DEVICES

(75) Inventor: Paul H. Wang, Honolulu, HI (US)

(73) Assignee: GEORGETOWN UNIVERSITY, Washington, DC (US)

(21) Appl. No.: 13/319,987

(22) PCT Filed: May 10, 2010
(86) PCT No.: PCT/US10/34249

§ 371 Date: Nov. 10, 2011

Related U.S. Application Data
(60) Provisional application No. 61/177,535, filed on May 12, 2009.

(51) Int. Cl. A61F 5/14; A43B 7/14

(52) U.S. Cl. 36/88

(57) ABSTRACT

The present invention relates to orthotic devices and footwear. In particular, the present invention relates to orthotic devices comprising a wedge configured to be placed beneath a forefoot.

### REFERENCE TO RELATED APPLICATIONS

This is a non-provisional application which claims priority to U.S. Provisional Application No. 61/177,535 filed on May 12, 2009, which is incorporated herein by reference in its entirety.

### FIELD OF THE INVENTION

The present invention relates to orthotic devices and footwear. In particular, orthotic devices comprising wedges configured to be placed beneath a forefoot.

### BACKGROUND

The ankle foot complex is designed to withstand numerous stresses. When running, ground reaction forces on the lower extremity increase thus placing the lower extremity under excessive stress. The foot and ankle complex has a role in providing a stable support for the body against the ground; absorbing shock; permitting the foot to conform to changing terrain; and acting as a mechanical lever to transfer muscle energy into the ground to assist forward locomotion. The foot can be classified into three compartments: the hindfoot (Calcaneus and Talus); the midfoot (Cuboid, Navicular and three cuneiform bones, lateral, middle and medial); and forefoot (5 metatarsal rays, 14 phalanges and 2 sesamoid bones). The big toe is the Hallux.

During locomotion, movement of the foot, ankle and leg operate together as a complex motion. These movements include the sagittal plane movement which occurs at the talocrural joint and includes dorsiflexion (Extension) with an average range of 20°, and plantar flexion (Flexion) with an average range of 45°. The frontal (coronal) plane movement occurs at the subtalar joint and includes an inversion with an average range of 20°, and an eversion with an average range of 10°. The transverse plane movement occurs as the result of tibal or femoral rotation; and gives information regarding the position of these bones and their associated joints. Pronation and supination are complex triplanar movements. Pronation incorporates movement of eversion, dorsiflexion and abduction. Supination incorporates movement of inversion, plantarflexion and adduction. Finally, the minimal range of hallux extension required at the 1st metatarsophalangeal joint is 65°.

The term “gait” is generally defined as the coordinated sequence of the various biomechanical movements of the lower limbs of a person undergoing locomotion. Gait is more typically described in terms of gait cycle due to the repetition of these movements during locomotion. For example, walking is a typical gait cycle and is used herein to describe the gait cycle.

Walking is divided into two phases. The first phase is the stance phase, which comprises the weight bearing portion of each gait cycle and is initiated by heel contact or heel-strike and ends with toe-off of the same foot. The second phase is the swing phase, which is initiated with toe-off and ends with heel-strike. Basically, the swing phase comprises the swinging of one limb to further locomotion while the contralateral limb remains grounded. The phrase “toe-off” refers to the instance of final contact between the toe and the floor. In normal gait, the point of final contact point between the toe and the floor generally occurs at the very front, bottom edge of the toe.

The stance phase comprises three segments, including (1) an initial double stance, (2) a single limb stance, and (3) a terminal double limb stance. The initial double stance segment accounts for approximately 10% of the gait cycle, as does the terminal double limb stance. The single limb stance accounts for a greater portion of the gait cycle, approximately 40%. As such, the stance phase accounts for a total of approximately 60% of the gait cycle, while the swing phase accounts for the remaining 40%.

The two limbs typically do not share the load equally during the double stance segments. Moreover, the load is typically fluctuating between limbs as gait progresses. During normal gait, ipsilateral swing temporally corresponds to single limb stance by the contralateral limb. If the velocity of gait is increased, variations begin to occur in the respective percentages of both the stance phase and the swing phase, and the duration of each aspect of the stance phase decreases until the walk becomes a run, in which case each of the double support periods are eliminated.

One gait cycle may be thought of in terms of a single stride. A stride may be defined as the distance between two successive placements of the same foot. Basically, a stride consists of two step lengths, left and right, each of which is the distance by which one foot moves forward in front of the other one. In normal gait, a person's step lengths are substantially similar to one another, whereas in pathological gait, or abnormal gait, it is possible for the two step lengths to differ.

More specifically, the gait cycle, or a single stride, comprises eight phases. The stance phase of the gait cycle comprises five sub-phases: (1) initial contact (the first 0-10% of the gait cycle), which occurs during initial double support and which includes initial contact, or heel-strike, and the loading response; (2) loading response (also within the first 0-10% of the gait cycle); (3) mid-stance (the next 10-30% of the gait cycle), which involves the progression of the body center of mass over the support foot and which trend continues through terminal stance; (4) terminal stance (the next 30-50% of the gait cycle), which begins with heel rise of the support foot and terminates with contralateral foot contact; and (5) pre-swing (the next 50-60% of the gait cycle), which begins with terminal double support and ends with toe-off of the ipsilateral limb. The swing phase of the gait cycle comprises the remaining three sub-phases: (1) initial swing (the next 60-73% of the gait cycle); (2) mid swing (the next 73-87% of the gait cycle); and (3) terminal swing (the remaining 87-100% of the gate cycle), each of which collectively effect foot clearance and advancement of the trailing limb.

To allow walking the foot flexes during the initial stages of the stance phase. This flexibility allows the foot to accommodate the uneven surfaces of the ground. To achieve this flexibility the foot is typically in an open-packed position. Plantar flexion of the talocrural joint equates to an open-packed foot and ankle. During the push off position the foot becomes stiff and stable to propel the foot forward. This is a foot in a closed packed position. The dorsiflexion of the talocrural joint locks the talus into the mortice of the tibia and fibula. The windlass mechanism of the plantar fascia contributes to the stability of the foot by stabilizing the arches of the foot. The windlass mechanism occurs during toe off where the metatarsophalangeal joints extend and pull the plantar fascia taut. This tension in the plantar fascia assists in stabilization of the longitudinal arch at toe off and provides a more rigid foot.

### SUMMARY

Embodiments of the invention relate to orthotic devices comprising wedges configured to be placed beneath a forefoot. Some embodiments include an orthotic device comprising a wedge configured to be placed beneath a forefoot, wherein the wedge comprises an upper surface, a lower surface, a front surface, a rear surface, wherein the gradient between the upper surface and the lower surface comprises an angle increasing from the outside surface of the wedge to the inside surface of the wedge.

In some embodiments, the thickest part of the device is beneath the first metatarsal and proximal phalanx joint of the forefoot.

In some embodiments, the gradient comprises an angle of about 1, 2, 3, 4, 5, 6, 7, 8, 9, or 10 degrees.

In some embodiments, the front surface is beveled. In some embodiments, the rear surface is beveled.

In some embodiments, the upper surface is substantially planar. In more embodiments, the upper surface is substantially convex. In further embodiments, the upper surface is substantially concave. In even more embodiments, the upper surface is stepped.

In some embodiments an orthotic device comprising a wedge configured to be placed beneath a forefoot is adapted to fit inside a shoe.

In some embodiments, the device is an integral part of a shoe.

In some embodiments an orthotic device comprising a wedge configured to be placed beneath a forefoot is adapted to fit underneath the sole of a shoe.

In some embodiments, the shoe is a spike shoe.

In some embodiments an orthotic device comprising a wedge configured to be placed beneath a forefoot, further comprises spikes.

In some embodiments an orthotic device comprising a wedge configured to be placed beneath a forefoot is adapted to fit in a ballet shoe.

In some embodiments an orthotic device comprising a wedge configured to be placed beneath a forefoot can be attached to the forefoot of a user.

Some embodiments include kits for using an orthotic device comprising a wedge configured to be placed beneath a forefoot and instructions for using the wedge.

Some embodiments include methods for treating forefoot varus comprising: identifying a subject in need thereof; adapting a wedge configured to be placed beneath a forefoot to fit within a shoe of said subject; and inserting said wedge into said shoe.

### BRIEF DESCRIPTION OF THE DRAWINGS

FIG. 1A is a perspective view of an orthotic device comprising a wedge. FIG. 1B is a perspective view of an orthotic device having beveled edges. FIG. 1C is a perspective view of an orthotic device having beveled edges.

FIG. 2A is a side view of an orthotic device having a planar surface. FIG. 2B is a side view of an orthotic device having a concave surface. FIG. 2C is a side view of an orthotic device having a stepped surface. FIG. 2D is a side view of an orthotic device having a convex surface.

FIG. 3A is a top view of a foot depicting a plurality of bones in the foot, and an orthotic beneath the foot. FIG. 3B is a side view of the orthotic device of FIG. 3A taken along the line 3A-3B.

FIG. 4 is a perspective view of a foot and an orthotic device underneath a portion of the foot.

FIG. 5 is a perspective view of a foot and an orthotic device underneath a portion of the foot, with the orthotic device having beveled edges.

FIG. 6A is a plan view of an orthotic device having beveled edges for use beneath a right foot. FIG. 6B is a side view of the orthotic device of FIG. 6A taken along the line 6C-6D. FIG. 6C is a side view of the orthotic device of FIG. 6A taken along the line 6A-6B

FIG. 7 is a plan view of a right foot and the orthotic device of FIG. 6 underneath a foot.

FIG. 8 is a side view of a show having an orthotic device inserted in the shoe.

FIG. 9 is a side view of a show having an orthotic device where the orthotic device is attached to the sole of the shoe.

FIG. 10 is a side view and front view of an orthotic device.

FIG. 11 shows a spike shoe (left), and an orthotic device adapted to fit in the spike shoe (right).

### DETAILED DESCRIPTION

Embodiments relate to orthotic devices. Some embodiments can include a wedge configured to be placed beneath a forefoot, wherein the wedge comprises an upper surface, a lower surface, a front surface, a rear surface, wherein the gradient between the upper surface and the lower surface comprises an angle increasing from the outside surface of the wedge positioned under the 5^{th} metatarsal to the inside surface of the wedge positioned under the 1^{st} metatarsal.

In some embodiments, the orthotic devices described herein can include inserts adapted to fit inside a shoe. Such shoes can include, for example. running shoes, track shoes, spikes, track spikes, and dance shoes such as ballet slippers, ballet flats, and ballet pointes. The device can be disposable. In some embodiments, the device can be an integral component of a shoe, for example, an insole, a sole, or a spike plate. In more embodiments, an orthotic device described herein can be attached to the forefoot of a user or attached to a sock liner. Some embodiments include kits comprising an orthotic device adapted to fit inside a shoe, or an orthotic device adapted to fit on the undersole of a shoe.

The orthotic devices described herein can be used to improve various forms of gait. For example, the orthotic devices described herein can be used to treat forefoot varus. In some embodiments, the orthotic devices described herein improve aspects of gait, such as running. Particular types of running include sprinting. Examples of sprints include track events. Examples of track events are well known and include the 50 m, 55 m, 60 m, 100 m, 200 m, 400 m, 800 m, 1000 m. 1500 m, 1600 m, one mile, 55 m with hurdles, 60 m with hurdles, 110 m with hurdles, and 400 m with hurdles.

In contrast to normal gait mechanics of walking, running removes the heel strike and the mid-stance component of the gait cycle. In order to most efficiently propel the body in running the foot must remain a rigid lever with only the forefoot striking the ground. Runners achieve higher speeds with greater force delivered during the forefoot strike and shorter total foot ground contact time.

Abnormalities in rear foot, mid-foot, and forefoot positioning as well as excessive instability in normal gait creates inefficiencies in the gait cycle as well as the possibility for greater risk of injuries. These abnormalities also decrease the efficiency and ability for runners to create a rigid lever to propel themselves. However, as the forefoot is the primary contact point with the ground for sprinters, misalignment of the forefoot can create suboptimal joint alignment and subsequent ability to stabilize joints throughout the lower extremity kinetic chain.

One aspect of the present invention recognizes that footwear, such as track spikes and dance shoes lack internal volume to accommodate bulky orthotic devices. Another aspect recognizes that it is the forefoot of a sprinter that contacts the ground during sprinting, and while the sprinter may rely on intrinsic muscles to stabilize the mid and rear foot, the forefoot may still require adjustment.

Without wishing to be bound by any one theory, the orthotic devices described herein can be useful to increase the force a foot strikes the ground during running, such as sprinting. Human sprinters normally take longer strides than those of non-sprinters. One way of achieving longer strides may be to apply great forces to the ground. At any speed, applying greater forces in opposition to gravity should increase a runner's vertical velocity on takeoff, thereby increasing both aerial time and forward distance traveled between steps. The first toe (hallux) may carry approximately 50% of the force transferred by a runner to the ground. In some embodiments, the orthotic devices described herein reduce the time taken for the first toe to contact the ground, thus increasing the total force transmitted through the first toe as the toe strikes the ground.

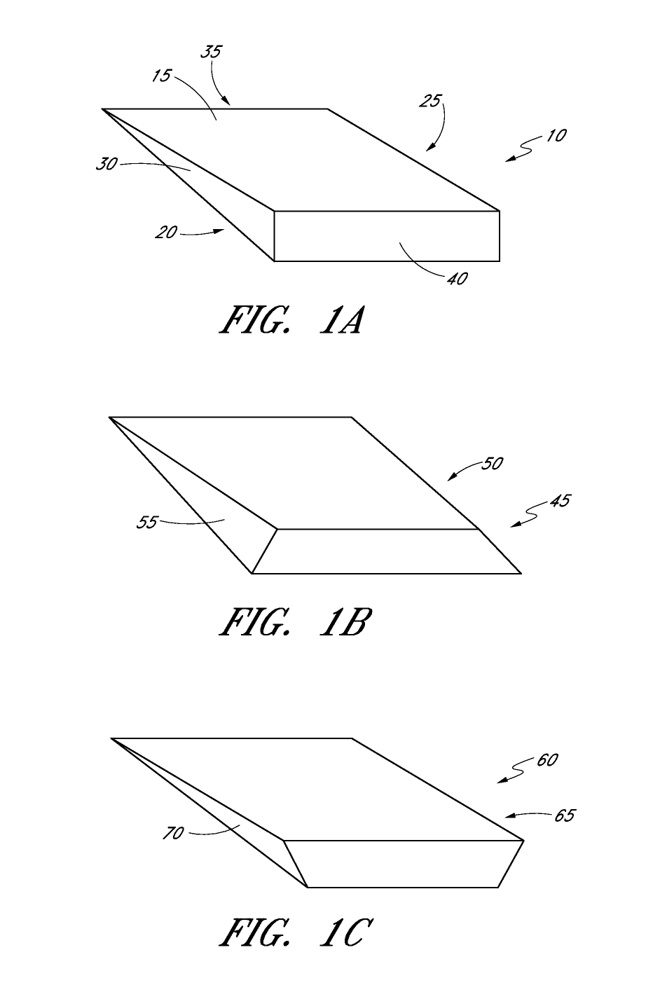

FIG. 1 shows some embodiments of orthotic devices. FIG. 1A is a perspective view of an orthotic device for a left foot comprising a wedge 10. The wedge 10 comprises an upper surface 15, a lower surface 20, a front surface 25, a rear surface 30, an outside surface 35, and an inside surface 40. The upper surface 15 is substantially smooth and can be coated with a layer to increase comfort and/or friction between the device and the forefoot Examples of coatings include materials such as moleskin. The lower surface 20 can be coated with a material to attach the wedge to a shoe, such as an adhesive. FIG. 1B is a perspective view of an orthotic device for a left foot comprising a wedge 45 having a beveled front surface 50 and a beveled rear surface 55. Some embodiments can include wedges with a beveled front surface and/or a beveled rear surface. As FIG. 1C illustrates with a perspective view of an orthotic device for a left foot comprising a wedge 60 having a beveled front surface 65 and a beveled rear surface 70, bevels can be in at least an upper-lower surface orientation, or a lower-upper surface orientation. Orthotic devices comprising any orientation of beveling are contemplated where such devices can be adapted to fit in the shoe of a user. Beveling can increase the fit of an orthotic device in a shoe, and can increase comfort. In some embodiments, beveling can ensure a tight fit at the front a shoe between the device and inside surface of the shoe. Such fits are preferred in order to transmit forces directly from the front of the shoe to the foot of the wearer.

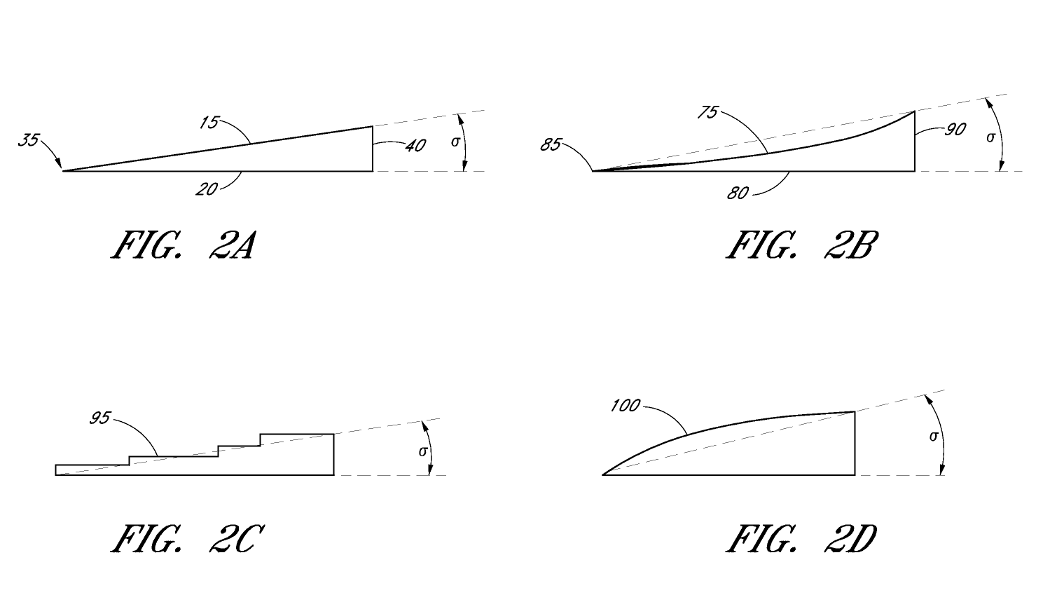

FIG. 2A is a side view of an orthotic device having a lower surface 20, outside surface 35, inside surface 40, and having an upper surface 15 having a planar aspect 75. The upper surface and lower surface are separated by the angle θ. The angle θ can be about 1, 2, 3, 4, 5, 6, 7, 8, 9, or 10 degrees or more. FIG. 2B is a side view of an orthotic device having an upper surface with a concave aspect 75, a lower surface 80, an outside surface 85, and inside surface 90. The upper surface and lower surface are separated by the angle θ. The angle θ can be about 1, 2, 3, 4, 5, 6, 7, 8, 9, or 10 degrees or more. FIG. 2C shows another embodiment of an orthotic device having an upper surface with a stepped aspect 95. FIG. 2D shows another embodiment of an orthotic device having an upper surface with a convex aspect 100. As will be appreciated, wedges can include an upper surface with a combination of ridges, troughs, and steps so to adapt for comfort and fit to the forefoot of a wearer.

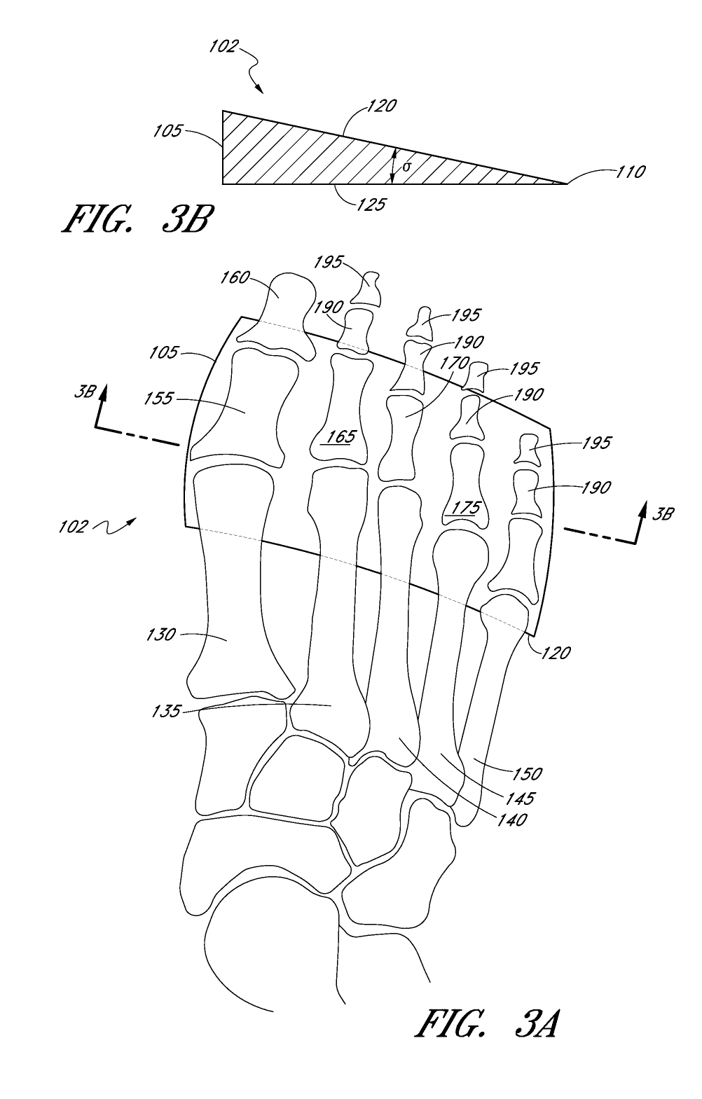

FIG. 3A is a top view of a right foot depicting a plurality of bones in the foot, and an orthotic device beneath the foot. The orthotic device 102 comprises an inside surface 105, an outside surface 110, a front surface 115, and a rear surface 120. FIG. 3B is a side view of the orthotic device of FIG. 3A taken along the line 3A-3B, comprising a lower surface 125 and upper surface 120 separated by the angle θ. As can be seen from FIG. 3a, in some embodiments, the orthotic device is placed beneath the forefoot of a wearer. The forefoot can include the distal portions of the 1^{st} metatarsal 130, 2^{nd} metatarsal 135, 3^{rd} metatarsal 140, 4^{th} metatarsal 145, and 5^{th} metatarsal 150, the proximal phalanx 155, the hallux 160, and the 2^{nd} proximal phalange 165, 3^{rd} proximal phalange 170, 4^{th} proximal phalange 175, and 5^{th} proximal phalange 180, intermediate phalanges 190 and distal phalanges 195. In some embodiments, the orthotic device is positioned with the thickest point of the wedge beneath the distal portion of the 1^{st} metatarsal 130 and proximal phalanx 155, the device can further extend underneath the proximal portion of the hallux 160. In some embodiments, the orthotic device is positioned with the thickest point of the wedge beneath the distal portion of the 1^{st} metatarsal 130 and proximal phalanx 155, the device can further extend underneath the proximal portion of the hallux 160, and further extend underneath the distal portions of the 2^{nd} metatarsal 135, 3^{rd} metatarsal 140, 4^{th} metatarsal and 5^{th} metatarsal, and underneath the 2^{nd} proximal phalange 165, 3^{rd} proximal phalange 170, 4^{th} proximal phalange 175, and 5^{th} proximal phalange 180, and underneath at least the proximal portions of the intermediate phalanges 190.

As will be apparent, orthotic devices described herein can be designed to raise the joint between the 1^{st} metatarsal 130 and proximal phalanx 155 to a greater extent than other metatarsal-phalange joints of the forefoot. In one embodiment, the orthotic device can be designed to raise the joint between the 1^{st} metatarsal 130 and proximal phalanx 155 to a greater extent than other metatarsal-phalange joints of the forefoot, and to raise the joint between the proximal phalanx 155 and hallux 160 to a greater extent than the joints between the intermediate phalanges 190 and distal phalanges 195.

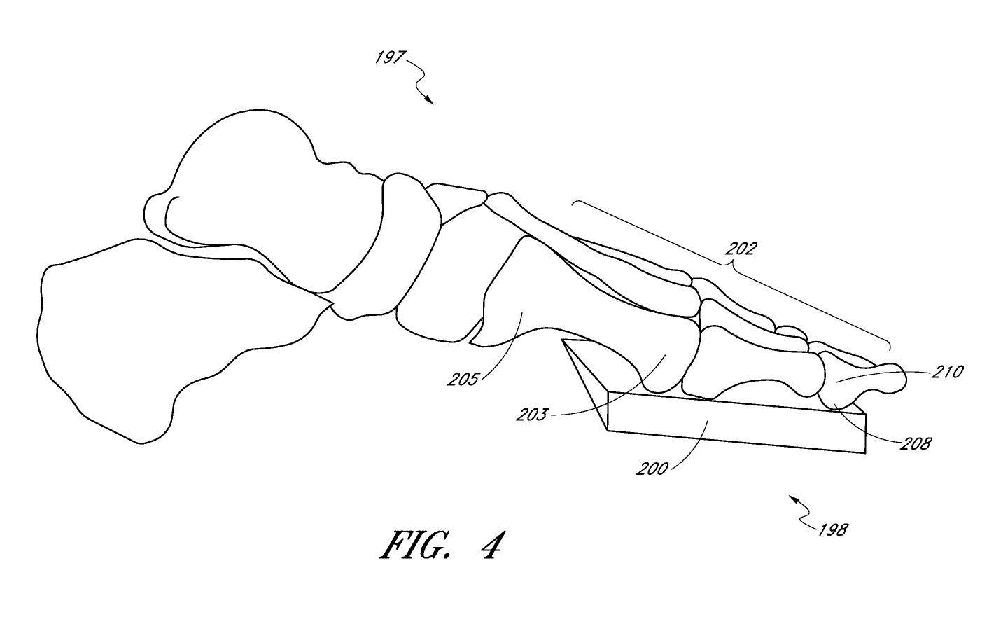

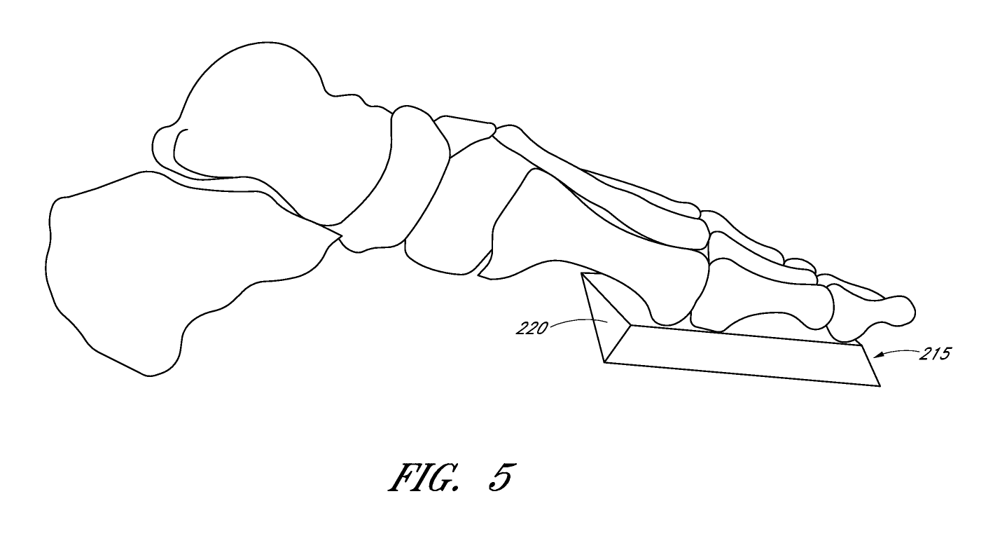

FIG. 4 is a perspective view of a left foot 197 and an orthotic device 198 comprising a wedge 200 underneath a portion 202 of the foot. The wedge extends from the distal portion 203 of the 1^{st} metatarsal 205 to the proximal portion 208 of the hallux 210, and underneath the other metatarsal joints of the foot. FIG. 5 shows a perspective view of a foot and an alternate embodiment of an orthotic device underneath a portion of the foot, with the orthotic device having a beveled front surface 215 and a beveled rear surface 220.

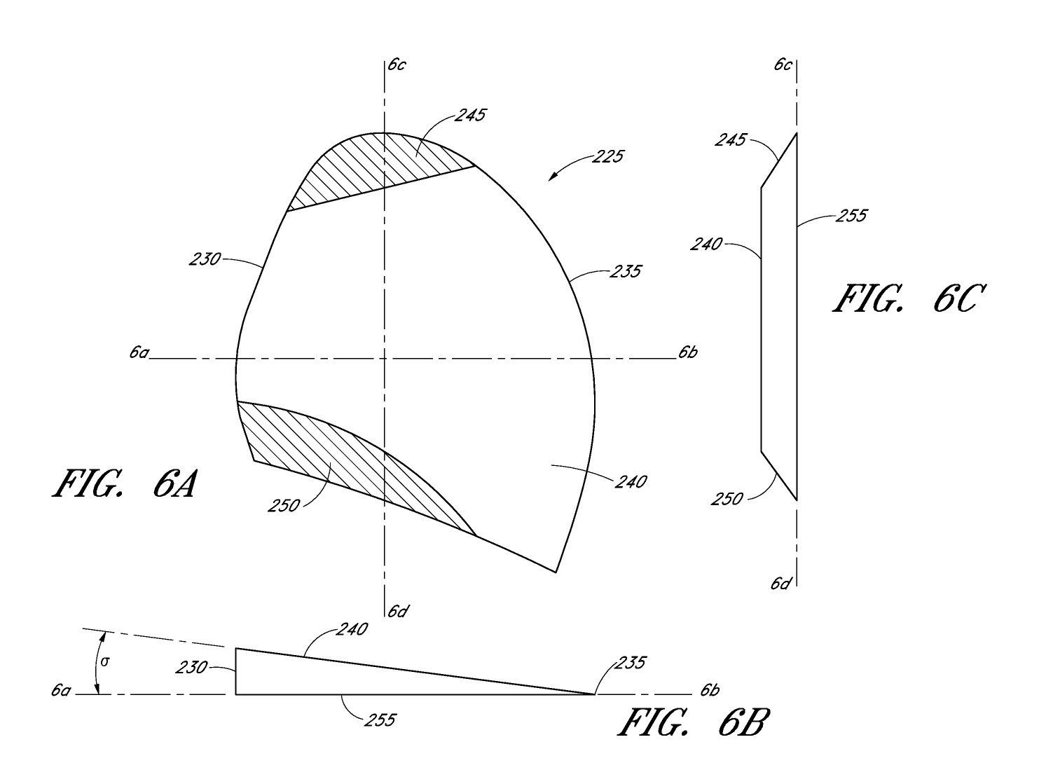

FIG. 6A is a plan view of an orthotic device comprising a wedge 225 having an inside surface 230, outside surface 235, upper surface 240, beveled front surface 245, and beveled rear surface 250. The orthotic device is adapted to fit in a shoe and configured to extend beneath the forefront of the wearer. FIG. 6B is a side view of the orthotic device of FIG. 6A taken along the line 6C-6D showing the lower surface 255 of the orthotic device. FIG. 6C is a side view of the orthotic device of FIG. 6A taken along the line 6A-6B. The upper surface 240 and lower surface are separated by the angle θ. The angle θ can be about 1, 2, 3, 4, 5, 6, 7, 8, 9, or 10 degrees.

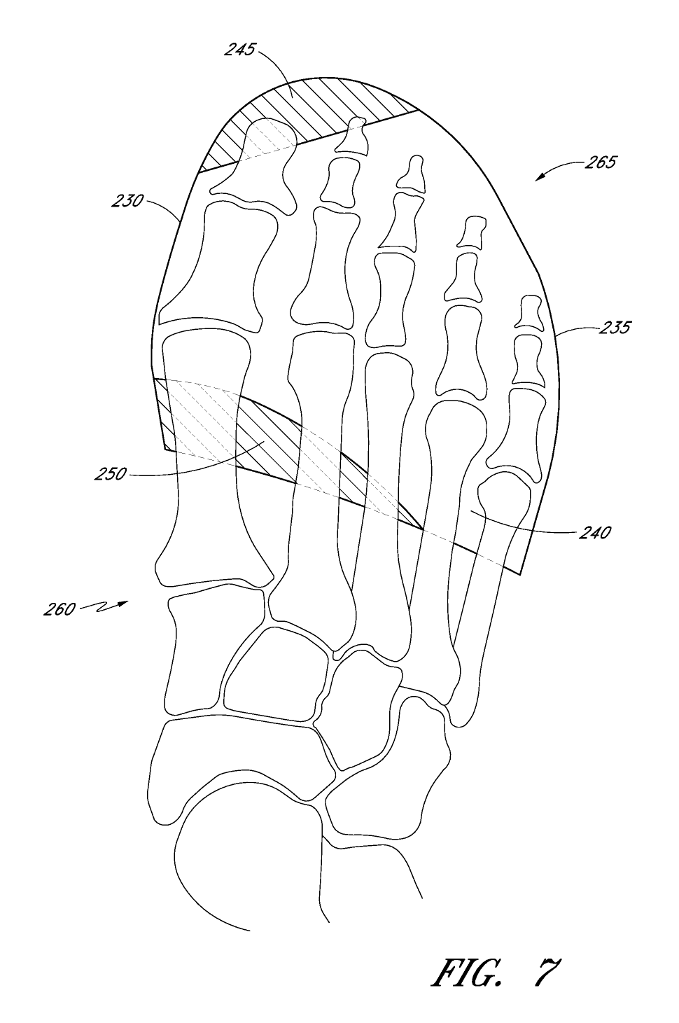

It is anticipated that the orthotic devices described herein are adapted to fit in a shoe and configured to extend beneath the forefoot of the wearer. One embodiment of an orthotic device configured to fit beneath the forefront of a wearer is shown in FIG. 7. FIG. 7 is a plan view of a left foot 260 and the orthotic device 265 of FIG. 6 underneath a right foot.

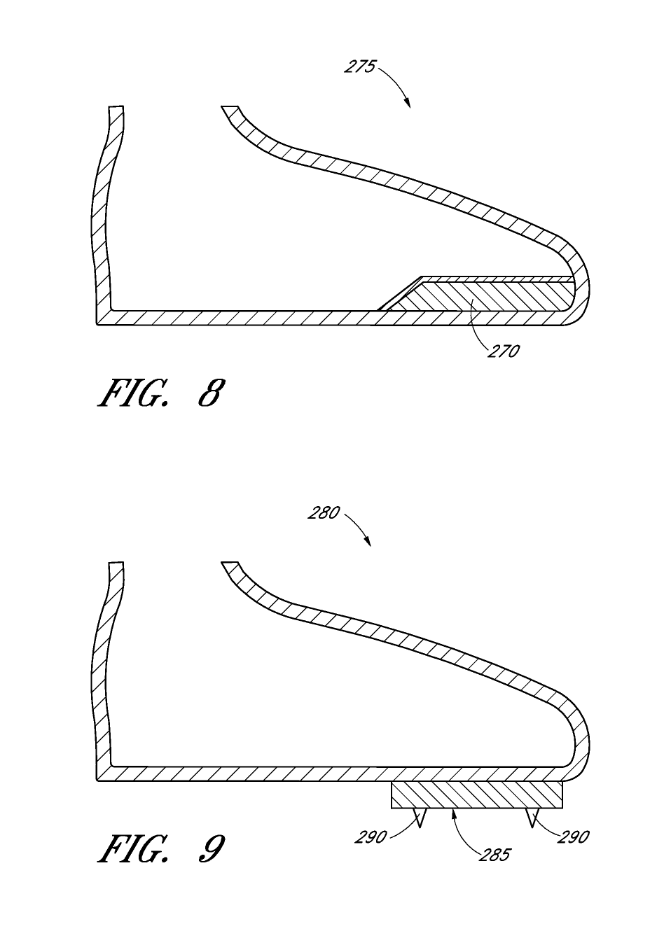

The orthotic devices described herein can be inserts adapted to fit in the shoe of a wearer. FIG. 8 is a side view of a shoe 275 having an orthotic device 270 inserted in the shoe. The orthotic device can extend to any portion of the foot. For example, the orthotic device can include an insole that extends the length of the underside of the foot.

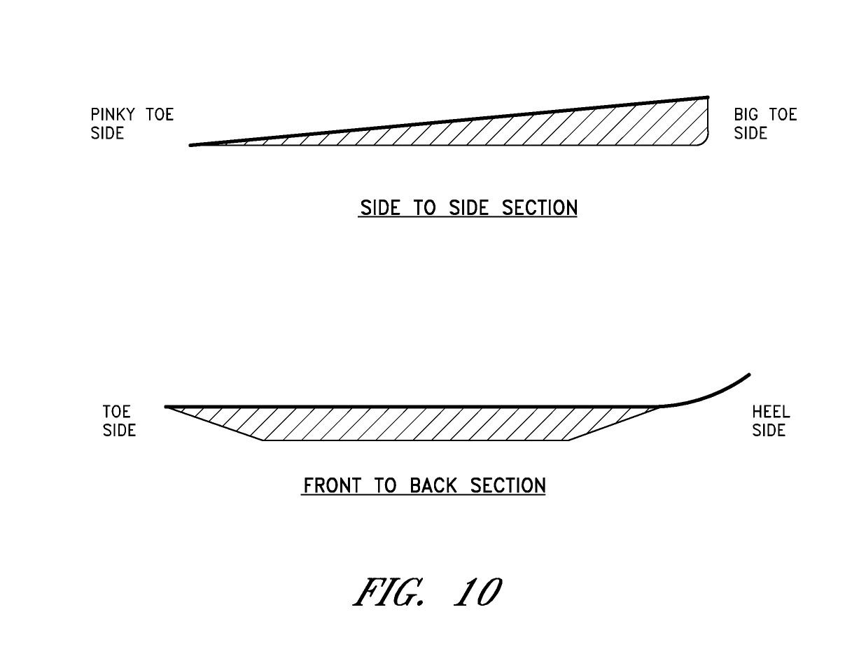

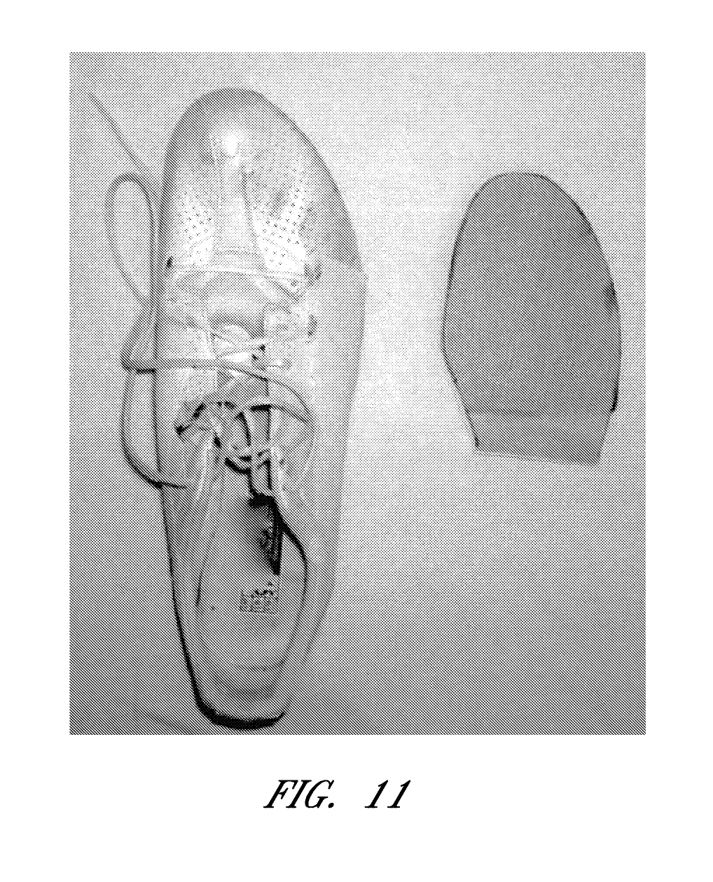

In some embodiments, orthotic devices can be an integral portion of a shoe, for example, an insole, a sole, a spike plate. FIG. 9 is a side view of a shoe 280 having an orthotic device 285 where the orthotic device is attached to the sole of the shoe and further comprises spikes 290.

Some embodiments include kits comprising an orthotic device and instructions for use. Such devices may be provided to be adapted by a user to fit a shoe. For example, an orthotic device can be provided in a form where a user will adapt the orthotic device to fit within a shoe and to be configured to be positioned underneath the forefoot of a user. The orthotic device can include markings to indicate how the device can be adapted for different sizes of feet. In more embodiments, a kit can comprise an orthotic device that can be adapted on the lower surface of the sole of a shoe. In some such embodiments, the orthotic device can comprise a spike plate. Such spike plates can receive spikes.

The orthotic devices described herein can comprise any material known in the art. The material can be compressible and resilient to provide cushioning and resistance. The material can have open-cells. Examples of materials include thermoplastics, polyethylene, polypropylene, ethylene vinyl acetate (EVA), UCOLITE, cork, rubber, and gels (U.S. Pat. No. 7,105,607, hereby incorporated by reference in its entirety).

The devices described herein can be provided with an adhesive layer to position and secure the device under the foot at a desired location. Any suitable adhesive known in the art may be employed. However, it should be noted that if the device is intended to be applied directly to the foot, a non-irritating adhesive should be used. In one embodiment, the adhesive layer may be applied to the upper surface of the device, such that the device may be secured directly to the foot of a wearer or to the underside of the sock liner of footwear at the desired location. Alternatively, the adhesive layer may be applied to the lower surface of the device, such that the device may be secured to the upper-side of the sock liner of footwear, or to the insole of footwear at the desired location.

Some embodiments include methods for treating forefoot varus. Such embodiments can include identifying a subject in need of treatment, and adapting an orthotic device described herein to fit underneath the forefoot of the subject. In some embodiments, the orthotic device can be adapted to fit within a shoe of the subject and configured to be placed beneath the forefoot of the subject. Methods for treating forefoot varus can further include inserting an orthotic device into the shoe of the subject, and/or attaching the orthotic device to the subject.

More embodiments include methods for making the orthotic devices described herein. Some such methods can include configuring a material to fit underneath a forefoot, for example, by shaping a material. Some methods for making the orthotic devices described herein can further include adapting a material to fit inside a shoe. Shaping can be performed by a variety of methods, for example, cutting a material to fit, molding a material to fit, and grinding a material to fit. More methods can include applying layers to the device, such layers can include an adhesive layer to position and secure the device under the foot at a desired location.

More embodiments can include methods for improving the efficiency in the gait of a sprinter sprinting. Such methods can include placing an orthotic device described herein underneath the forefoot of a sprinter. Such methods can further include providing an orthotic device described herein to a sprinter, and/or measuring an increase in efficiency in the gait of the sprinter sprinting. Measuring an increase in the efficiency in the gait of a sprinter can be performed by a variety of methods. For example, the excessive or aberrant motion of the foot ankle complex of a track sprinter without the orthotic may be analyzed using slow-motion video analysis and compared to the motion of the foot ankle complex of the same sprinter with the orthotic. A decrease in the aberrant or excessive motion of the foot ankle complex using the orthotic would indicate increased efficiency.

### EXAMPLE

An orthotic device described in FIGS. 6 and 7 was inserted into each track spike. A sprinter wore the track spikes. While the sprinter ran, an increase in the amount of surface area that the forefoot of the sprinter contacted the ground at the time of initial forefoot/ground contact was observed, compared to the sprinter not wearing the orthotic device. While the sprinter ran, a reduction in the lateral motion of the foot was observed, compared to the sprinter not wearing the orthotic device. A decrease in forefoot varus of the sprinter wearing the orthotic device was observed, compared to a sprinter not wearing the orthotic device.

It will be apparent to those skilled in the art that some modifications and variations of the present invention can be made without departing form the spirit and scope of the invention. Thus, it is intended that the present invention cover the modifications and variations of this invention provided they come within the scope of the claims and their equivalents.

What is claimed is:

1. An orthotic device comprising a wedge configured to be placed beneath a forefoot, wherein said wedge comprises an upper surface, a lower surface, a front surface, a rear surface, wherein the gradient between said upper surface and said lower surface comprises an angle increasing from the outside surface of said wedge to the inside surface of said wedge.

2. The device of claim 1, wherein the thickest part of said device is beneath the first metatarsal and proximal phalanx joint of said forefoot.

3. The device of claim 1, wherein said gradient comprises an angle of about 4 degrees.

4. The device of claim 1, wherein said front surface is beveled.

5. The device of claim 1, wherein said rear surface is beveled.

6. The device of claim 1, wherein said upper surface is substantially planar.

7. The device of claim 1, wherein said upper surface is substantially convex.

8. The device of claim 1, wherein said upper surface is substantially concave.

9. The device of claim 1, wherein said upper surface is stepped.

10. The device of claim 1 adapted to fit inside a shoe.

11. The device of claim 1, wherein said device is an integral part of a shoe.

12. The device of claim 1 adapted to fit underneath the sole of a shoe.

13. The device of claim 10 wherein said shoe is a spike shoe.

14. The device of claim 13, further comprising spikes.

15. The device of claim 10 wherein said shoe is a ballet shoe.

16. The device of claim 1 attached to the forefoot of a user.

17. A kit for using an orthotic device comprising a wedge configured to be placed beneath a forefoot and instructions for using said wedge.

18. A method for treating forefoot varus comprising:
identifying a subject in need thereof;
adapting a wedge configured to be placed beneath a forefoot to fit within a shoe of said subject; and
inserting said wedge into said shoe.
